(19) United States
(12) Patent Application Publication
(10) Pub. No.: US 2006/0293359 A1
(43) Pub. Date: Dec. 28, 2006

(54) Methods and compositions for the treatment of diabetes

(75) Inventors: Jyotirmoy X. Kusari, Irvine, CA (US); Daniel W. Gil, Corona Del Mar, CA (US); John E. Donello, Dana Point, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/460,386

(22) Filed: Jul. 27, 2006

Related U.S. Application Data
(63) Continuation-in-part of application No. 10/891,740, filed on Jul. 15, 2004.
(63) Continuation-in-part of application No. 10/607,439, filed on Jun. 25, 2003.
(63) Continuation-in-part of application No. 10/891,953, filed on Jul. 15, 2004.
(60) Provisional application No. 60/502,840, filed on Sep. 12, 2003.
(60) Provisional application No. 60/502,562, filed on Sep. 12, 2003.

(51) Int. Cl. A61K 31/4709; A61K 31/4168

(52) U.S. Cl. 514/314; 514/389

(57) ABSTRACT

Disclosed is a method for treating symptoms of diabetes using an agonist of the α2B and/or α2C adrenergic receptor subtypes that lacks (a) significant α2A adrenergic receptor activity or (b) significant α1A adrenergic receptor activity, or that lacks both (a) and (b).

The present patent application is a continuation-in-part of co-pending U.S. patent application Ser. No. 10/891,740, filed Jul. 15, 2004, which claims priority to U.S. Provisional Application No. 60/502,840, filed Sep. 12, 2003, and is also a continuation-in-part of co-pending U.S. patent application Ser. No. 10/607,439, filed Jun. 25, 2003, and is a continuation-in-part of U.S. patent application Ser. No. 10/891,953, filed on Jul. 15, 2004, which claims priority to U.S. Provisional Application No. 60/502,562, filed Sep. 12, 2003, All of the foregoing application are incorporated by reference herein.

### FIELD OF THE INVENTION

The present invention is directed to methods of treating symptoms of diabetes using an agonist of the α2B and/or α2C adrenergic receptor subtypes that lacks (a) significant α2A adrenergic receptor activity or (b) significant α1A adrenergic receptor activity, or that lacks both (a) and (b).

### BACKGROUND AND SUMMARY

Diabetes mellitus is a condition involving the presence of abnormally high levels of glucose in the blood (hyperglycemia). A normal range of glucose in the blood is considered between about 70 mg/dl and about 110 mg/dl. Hyperglycemia would comprise a blood glucose level above 110 at a time greater than about 2-3 hours after eating. This condition arises due to reduced or absent production or secretion of insulin (Type 1 or insulin-dependent diabetes), or to a cell's lack of response to the presence of insulin in the extracellular milieu (Type 2 or insulin independent diabetes). Type-2 insulin resistant diabetes mellitus accounts for 90-95% of all diabetes, and affects approximately 6% of adults in Western societies. The incidence of the disease is growing worldwide at a rate of 6% per year. Characteristic features include insulin resistance, hyperglycemia, hyperlipidemia, obesity, and hypertension.

Insulin is made in the pancreas by β islet cells. Normally, insulin is released by the pancreas following absorption of glucose into the bloodstream after a meal. Most cells of the body have insulin receptors (IR) on their cell membranes; when the insulin receptor binds circulating insulin, a complex chain of events is initiated.

The IR is a tetrameric protein comprised of two alpha subunits (135 kDa each) and two beta subunits (95 kDa), which are linked together by disulfide bonds. The alpha-subunits are entirely extracellular, and the beta-subunits cross the plasma membrane. Insulin binding occurs entirely through contacts made to the alpha-subunits, however the intracellular portion of the beta-subunit is also essential for insulin action.

The IR has been discovered to be a tyrosine kinase, and the effects of insulin binding include translate to specific phosphorylation through signal transduction pathways leading to an increase in intracellular storage of glucose and a decrease in the amount of hepatic glucose release.

Increase in intracellular storage of glucose in adipocytes and skeletal muscle is accomplished partly by insulin-dependent increase in the recruitment of glucose transporter molecules (GLUTs) to the plasma membrane; these transporters (GLUT 4) exist in cells of adipose and skeletal muscle tissues as a pool of transporter molecules sequestered in a pool in the cytoplasm. Insulin-dependent activation of protein kinase B (PKB) and protein kinase C-1 (PKC-1) cause the migration of glucose transporters from their intracellular location to the plasma membrane. Skeletal muscle and adipocytes account for about 95% of glucose uptake.

In the liver, insulin binding triggers an increase in glucose uptake due to the increased activity of the enzymes glucokinase, phosphofructokinase-1, and pyruvate kinase, which regulate glycolysis to a major extent. Other insulin-binding dependent phosphorylation events result in a net increase in intracellular glucose in hepatocytes and a reduced blood glucose level. At the same time, insulin stimulates glycogen synthetic enzyme expression and activity. Insulin binding to the IR also has a significant effect on the transcription of certain genes involved in glucose and fatty acid metabolism.

Epinephrine has been noted to diminish insulin secretion by islet , cells by employing a cAMP-mediated regulatory pathway. Epinephrine functions in a manner opposite to that of insulin in liver and peripheral tissue. Epinepherine binding to β-adrenergic receptors, inducing adenylate cyclase activity, increasing the intracellular concentration of cAMP and activating Protein Kinase A (PKA) in a manner similar to glucogon, whose activity opposes that of insulin. The increase in PKA activity and in cAMP induces glycogenolysis and gluconeogenesis. These events result in an increase in blood glucose levels and thus counters insulin's effect in lowering the concentration of blood glucose.

Epinephrine also influences glucose homeostasis through an interaction with α adrenergic receptors. These receptors, members of the G-protein coupled receptor (GPCR) family, are associated with a receptor selective G-protein. Upon epinephrine binding, the G protein activates phospholipase C-γ (PLC-γ), which converts phosphoinositol bisphosphate (PIP₂) to the second messengers inositol triphosphate (IP₃) and diacylglycerol (DAG). IP₃ then stimulates the activation of calmodulin and initiates a series of phosphorylations, leading to inhibition of glycogen synthase, which blocks the incorporation of glucose into glycogen.

Thus, the α adrenergic receptors are known to influence glucose metabolism in certain ways. These receptors are components of the sympathetic branch of the autonomic nervous system, which is a relatively independent and involuntary branch of the nervous system. The autonomic nervous system, which innervates organs including the eyes, the lacrimal gland, the submandibal and sublingual glands, the parotid gland, the heart, trachea, liver, stomach, small intestine, adrenal medulla, kidneys, large intestine, bladder and uterus, is further divided into the sympathetic and parasympathetic nervous systems.

The nerves of the parasympathetic division have ganglia located in the organs that they innervate. These ganglia obtain neural inputs from fibers arising from cells in certain nuclei of the brainstem and sacral spinal cord. The fibers innervating a ganglion are defined as preganglionic and those arising from it are called postganglionic.

By contrast, the sympathetic ganglia are arranged in a cord along the vertebral column or in the mesentery of the gut. The output cells have long postganglionic fibers that branch and innervate the internal organs. These output cells are innervated by preganglionic fibers of cells located in the intermediolateral column of the thoracolumbar portions of the spinal cord. A special subdivision of the sympathetic system is located in the interior of the adrenal gland. The chromaffin cells located within the aderenal medulla contain vesicles filled with catacholamines, such as epinephrine. When stimulated, the chromaffin cells discharge their contents into the bloodstream.

The ganglion cells in the sympathetic system have wide fields of innervation and their activity have wide spread effects. Traditionally it has been thought that the overall effect of the sympathetic system is to decrease activity in the viscera and to stimulate the heart and somatic muscles for fight or flight behavior. However, there are exceptions to this traditional view.

The sympathetic nervous system is known to be involved in the pathology of both obesity and diabetes, and is, in turn, affected by both of these conditions, which also tend to influence each other. For example, it has been hypothesized that in obesity there is stimulation of sympathetic outflow to the kidneys and to skeletal muscles. Vasoconstriction caused by the latter reduces glucose delivery and uptake in muscles, a hallmark of insulin resistance.

As mentioned above, the α and β adrenergic receptors are components of the sympathetic nervous system and respond to adreneline (epinephrine) or, more commonly, noradreneline (norepinephrine). Over stimulation of these receptors may play a key role in Type II diabetes. In previous studies, certain α2-receptor agonists, when contacted with pancreatic β-cells, mimicked the known effects of sympathetic nervous system stimulation, and inhibited insulin release, which result in an elevation of blood glucose. See e.g., Angel, et al., J. PHARM. EXP. THERAPEUTICS 254 877 (1990)(hereinafter “ANGEL”), Niddam et al., J. PHARM. EXP. THERAPEUTICS 254 883 (1990)(hereinafter “NIDDAM”), hereby incorporated by reference herein. Compounds tested in these studies were UK 14,304 (brimonidine), clonidine, p-aminoclonidine, oxymetazoline, epinephrine, norepinephrine and cirazoline. Each of these compounds, with the exception of cirazoline (an α1 selective agonist), were shown at a concentration of 0.3 μM in 20 mM glucose to inhibit glucose-stimulated insulin release from isolated pancreatic rate islets. The β-adrenoreceptor agonist isoproterenol failed to inhibit insulin release under similar circumstances. Experiments using α2B receptor antagonists (prazosin, ARC-239 and chloropromazine) and α1 antagonists (prazosin) appear to exclude the α1 and α2B receptor subtypes from involvement in the brimonidine-induced inhibition of insulin release. The alpha 2 agonists used in this study all have significant sedative and cardiovascular hypotensive activity. Id.

Other experiments have indicated that clonidine may act to reduce serum glucose. However, it is now not clear whether clonidine displays this activity through the α2 or I1 imidazole receptors; see e.g., Rocchini, et. al., HYPERTENSION, 33 (Part II): 548-553, January, 1999). The literature indicates that other α2 pan agonists lack hypoglycemic activity. By contrast moxonidine, which is a very selective I1 imidazole receptor agonist (with weak α2 activity) shows good anti-hyperglycemic activity. Since other α2 pan agonists having sedative activity are reported to have hyperglycemic activity rather than hypoglycemic activity, see ANGEL and NIDDAM, it is therefore not presently clear to the person of ordinary skill in the art whether clonidine exerts its hypoglycemic activity through the (α2 adrenergic receptors or the I1 imidazole receptors.

With the recent availability of a class of α2 receptor agonists lacking sedative activity it has become possible to study the effect of such agonists on glucose metabolism more closely. Examples of such compounds, methods of their making, and methods of screening such compounds are provided, for example and without limitation, in the following publications, all of which are incorporated herein by reference in their entirety: U.S. Pat. Nos. 6,329,369; 6,545,182; 6,841,684 and U.S. Patent Publications Serial No US20020161051, entitled “(2-hydroxy)ethyl-thioureas useful as modulators of alpha2B adrenergic receptors”; US20030023098, entitled “Compounds and method of treatment having agonist-like activity selective at alpha 2B or 2B/2C adrenergic receptors”; US20030092766, entitled “Methods and compositions for modulating alpha adrenergic receptor activity”; US20040220402, entitled “4-(substituted cycloalkylmethyl)imidazole-2-thiones, 4-(substituted cycloalkenylmethyl)imidazole-2-thiones, 4-(substituted cycloalkylmethyl)imidazol-2-ones and 4-(substituted cycloalkenylmethyl)imidazol-2-ones and related compounds”; US20040266776, entitled “Methods of preventing and reducing the severity of stress-associated conditions”; US20050059664 entitled “Novel methods for identifying improved, non-sedating alpha-2 agonists”; US20050059721, entitled “Nonsedating alpha-2 agonists”; US20050059744 entitled “Methods and compositions for the treatment of pain and other alpha 2 adrenergic-mediated conditions”; and US20050075366 entitled “4-(2-Methyl-5,6,7,8-tetrahydro-quinolin-7-ylmethyl)-1,3-dihydro-imidazole-2-thione as specific alpha2B agonist and methods of using the same”. Additional disclosure concerning non-sedating alpha 2 adrenergic agonists can be found in US20050058696, entitled Methods and Compositions for the Treatment of Pain and other Alpha 1 Adrenergic Mediated Conditions”, and US20040132824, entitled “Novel Methods and Compositions of Alleviating Pain”. All the patents and patent applications referenced above are incorporated by reference herein in their entirety.

These publications show that such non-sedating α2 receptor agonist compositions contain agents that have already been characterized in the imidazole, thiourea, imidazoline, and imidazole thione, amino imidazoline, amino oxazoline and amino thiazoline chemical classes. It is to be expected that future non-sedating α2 agents (or combinations of agents) will be found in additional chemical classes including phenethylamine, amino thiazine, benzazepine, quinazoline, guanidine, piperazine, yohimbine alkaloid, and phenoxypropanolamine chemical classes.

In particular, it has been found that non-sedating α2 adrenergic agonist compositions have certain biochemical properties in common, regardless of the chemical structure of the agents contained in the compositions. For example, in one embodiment such compounds, in addition to having α2 adrenergic agonist activity, particularly but not necessarily exclusively, α2B and or α2C adrenoreceptor activity, also lack significant al adrenoreceptor activity. However, in another embodiment, a therapeutic composition comprising a non-sedating α2 adrenergic agonist may comprise a combination of an α2 adrenergic agonist with an α1 adrenergic antagonist. In each case, the reduced or absent α1 adrenergic activity results in a significant increase in the potency of the α2 adrenergic agonist activity with no significant increase in the potency of the sedative activity. Thus, at therapeutically effective concentrations, the α2 adrenergic agonist has little or no sedative effect, particularly as compared to a composition comprising an α2 adrenergic agonist at a dosage conferring the same therapeutic effect, but lacking significant α1A inhibitory activity.

Potency, as used here, refers to the concentration of an agonist required to produce a therapeutic effect. Potency is quantified by EC₅₀, the concentration at which half of the maximum therapeutic effect of the agonist is seen. Change in potency, therefore, is quantified by a change in EC₅₀: an increase in potency, for example, results in a decrease in EC₅₀.

Efficacy, as used here, refers to maximum effect of an agonist. Percent efficacy (% E) is determined by comparing the maximum effect of each agonist to the maximum effect of a standard full agonist (phenylephrine for α-1 receptors and brimonidine for α-2 receptors.

By “lacking significant al A activity” is meant having an α1A/α2A EC₅₀ ratio greater than that of brimonidine (for which this ratio is greater than about 25). In preferred embodiments the ratio is at least 20% greater, or at least 40% greater, or at least 50% greater, or at least 70% greater, or at least 80% greater, or at least 100% greater, or at least 200% greater, or at least 500% greater than that of brimonidine.

In another embodiment, the non-sedating α2 adrenergic agonist may comprise a adrenergic agonist having selective α2B and/or α2C agonist activity, but lacking significant alpha 2A activity.

An “α2 agonist lacking significant α2A activity” is an α2 agonist that has less than 40% of the efficacy of brimonidine at the α2A receptor and has the ability to produce a therapeutic effect without concomitant sedation upon peripheral administration in genetically unaltered animals. It will be understood that such a characterization includes α2B selective agonists lacking significant α2A activity, α2C selective agonists lacking significant α2A activity, and α2B/α2C agonists lacking significant α2A activity. Such agonists have an EC₅₀ of less than 1000 nM at the indicated receptor subtype(s)(α2B, α2C, or α2B and α2C), or at least 100-fold greater activity at the indicated receptor subtype(s) than at the α2A receptor. Preferably, the agonists have an EC50 value of less than 900 nM, 800 nM, 700 nM, 600 nM, 500 nM, 400 nM, 300 nM, 200 nM, 100 nM, 50 nM, 10 nM or 1 nM at the indicated receptor subtype(s).

Agonist selectivity can be characterized using any of a variety of routine functional assays, for example, in vitro cell-based assays which measure the response of an agent proximal to receptor activation. Useful assays include, without limitation, in vitro assays such as cyclic AMP assays or GTPγS incorporation assays for analyzing function proximal to α2 receptor activation (Shimizu et al., J. NEUROCHEM. 16:1609-1619 (1969); Jasper et al., BIOCHEM. PHARMACOL. 55: 1035-1043 (1998); and intracellular calcium assays such as FLIPR assays and detection of calcium pulses by Ca⁺⁺-sensitive fluorescent dyes such as fluo-3 for analyzing function proximal to α1 receptor activation (Sullivan et al., METHODS MOL. BIOL. 114:125-133 (1999); Kao et al., J. BIOL. CHEM. 264:8179-8184 (1989)). α2A selectivity assays based on inhibition of forskolin-induced cAMP accumulation in PC 2 cells stably expressing an α2A receptor, and increases in intracellular calcium in HEK293 cells stably expressing an α2A receptor are known and have been described in, for example, U.S. Patent Application Publication No. 2005/0059664, which is incorporated by reference as part of this disclosure in its entirety. Additional useful assays include, without limitation, inositol phosphate assays such as scintillation proximity assays (Brandish et al., ANAL. BIOCHEM. 313:311-318 (2003)); assays for β-arrestin GPCR sequestration such as bioluminescence resonance energy transfer assays (Bertrand et al., J. RECEPTOR SIGNAL TRANSDUC. RES. 22:533-541 (2002)); and cytosensor microphysiometry assays (Neve et al., J. BIOL. CHEM. 267:25748-25753 (1992)). These and additional assays for α2 and α1 (for example α1A) receptor function are routine and well known in the art and are hereby incorporated by reference as part of this specification in their entirety.

As a non-limiting example, a GTPβS assay is an assay useful for determining, for instance, the functional selectivity of an agent for activating an α2A receptor as compared to an α1A receptor in the methods of the invention. α2 adrenergic receptors mediate incorporation of guanosine 5′-O-(γ-thio)-triphosphate ([³⁵S]GTPγS) into G-proteins in isolated membranes via receptor-catalyzed exchange of [³⁵S]GTPγS for GDP. An assay based on [³⁵S]GTPγS incorporation can be performed essentially as described in Jasper et al., supra, 1998. Briefly, confluent cells treated with an agent to be tested are harvested from tissue culture plates in phosphate buffered saline before centrifuging at 300×g for five minutes at 4° C. The cell pellet is resuspended in cold lysis buffer (5 mM Tris/HCl, 5 mM EDTA, 5 mM EGTA, 0.1 mM PMSF, pH 7.5) using a Polytron Disrupter (setting #6, five seconds), and centrifuged at 34,000×g for 15 minutes at 4° C. before being resuspended in cold lysis buffer and centrifuged again as above. Following the second wash step, aliquots of the membrane preparation are placed in membrane buffer (50 mM Tris/HCl, 1 mM EDTA, 5 mM MgCl₂, and 0.1 mM PMSF, pH 7.4) and frozen at −70° C. until used in the binding assay.

GTPγS incorporation is assayed using [³⁵S]GTPγS at a specific activity of 1250 Ci/mmol. Frozen membrane aliquots are thawed and diluted in incubation buffer (50 mM Tris/HCl, 5 mM MgCl₂, 100 mM NaCl, 1 mM EDTA, 1 mM DTT, 1 mM propranolol, 2 μM GDP, pH 7.4) and incubated with radioligand at a final concentration of 0.3 nM at 25° C. for 60 minutes. After incubation, samples are filtered through glass fiber filters (Whatman GF/B, pretreated with 0.5% bovine serum albumin) in a 96-well cell harvester and rapidly washed four times with four ml of ice-cold wash buffer (50 mM Tris/HCl, 5 mM MgCl₂, 100 mM NaCl, pH 7.5). After being oven dried, the filters are transferred to scintillation vials containing five ml of Beckman's Ready Protein® scintillation cocktail for counting. The EC₅₀ and maximal effect (efficacy) of the agent to be tested are then determined for the α2A receptor.

Various other methods can be used to assay receptor selectivity. For example, a method for measuring alpha agonist activity and selectivity comprises the RSAT (Receptor Selection and Amplification Technology) assay as reported in Messier et al., High Throughput Assays Of Cloned Adrenergic, Muscarinic, Neurokinin And Neurotrophin Receptors In Living Mammalian Cells, PHARMACOL. TOXICOL. 76:308-11 (1995), which has beenadapted for use with α1 and α2 receptors. The assay measures a receptor-mediated loss of contact inhibition that results in selective proliferation of receptor-containing cells in a mixed population of confluent cells. The increase in cell number is assessed with an appropriate transfected marker gene such as β-galactosidase, the activity of which can be easily measured in a 96-well format. Receptors that activate the G protein, G_{q}, elicit this response. Alpha 2 receptors, which normally couple to Gᵢ, activate the RSAT response when coexpressed with a hybrid Gq protein that has a Gᵢ receptor recognition domain, called Gq/i5. See Conklin et al., Substitution Of Three Amino Acids Switches Receptor Specificity Of G_{q}a To That Of Gᵢa, NATURE 363:274-6. (1993).

Using assay systems such as these, or other generally known methods, the person of ordinary skill in the art can screen drug libraries such as commercial drug libraries available from companies such as, without limitation, Sigma Aldrich, TimTec, Novascreen and the like to select compounds having α2 agonist activity, but lacking significant sedative activity at therapeutic concentrations of the drug.

Alternatively, known or unknown α2 agonists (such as the α2 pan agonist brimonidine) may be used in a non-sedating α2 agonist therapeutic composition comprising an α1 (preferably an α1A) antagonist to provide a therapeutic effect, wherein the dosage of the α2 agonist necessary to provide a therapeutic effect is substantially lowered in such composition relative to a second composition comprising only the α2 agonist as the sole active agent. Due to this increase in potency, the amount of sedation and cardiovascular depression experienced by a mammal to whom said agent is administered, either peripherally or non-peripherally, is greatly decreased at a therapeutically effective dose of the 2 agonist.

In specific embodiments of this non-sedating α2 adrenergic agonist composition, the al adrenergic receptor antagonist is selected from the group consisting of prazosin, terazosin, doxazonine, urapidil and 5-methylurapadil. The former two compounds and their syntheses are described in U.S. Pat. Nos. 3,511,836, and 4,026,894, respectively; the latter compound is an easily synthesized derivative of urapidil, whose synthesis is described in U.S. Pat. No. 3,957,786. These and all other references cited in this patent application are hereby incorporated by reference herein. Additionally, other al receptor antagonists (including α1A receptor antagonists) are well known in the art; many such compounds have been clinically approved. See also Lagu, 26 DRUGS OF THE FUTURE 757-765 (2001) and Forray et al., 8 EXP. OPIN. INVEST. DRUGS 2073 (1999), hereby incorporated by reference herein, which provide examples of numerous α1 antagonists.

The present invention is based in part on the surprising finding that α2-receptor agonist compositions are useful in treating hyperglycemia and hyperlipidemia and raising blood insulin levels, rather than in maintaining or causing hyperglycemia and hyperlipidemia, as has previously been observed in studies using α2 receptor agonist compounds having sedative activity. This effect is seen using non-sedating α2B selective receptor agonists compositions but is also observed using non-sedating α2 pan-agonist compositions as well.

By “pan-agonist” is meant that the agonist is α2 receptor agonist able to stimulate the α2A, α2B and α2C receptor subtypes.

By “α2 agonist composition” is meant that the composition comprises an α2 agonist having activity at the α2B and/or α2C adrenergic receptor subtypes, and either a) lacking significant α2A activity, b) lacking significant α1A activity, or both a) and b). In one embodiment the α2 agonist composition may comprise a non-sedating α2 receptor agonist, such as an α2 agonist lacking substantial α1A activity or an α2 agonist lacking significant α2A activity. In another embodiment the α2 agonist composition may comprise an α2 agonist (either an α2B or 2C selective agonist or an α2 pan-agonist) having activity at the α2B and/or α2C adrenergic receptor subtypes plus comprising an additional component selected from the group consisting of an α1 receptor antagonist (such as an α1A receptor antagonist) or an alpha 2A receptor antagonist or both.

The term “treat” means to deal with medically. It includes, for example, preventing the onset of a disease, alleviating its symptoms, or slowing its progression.

By a “therapeutically effective” amount, concentration, or dosage is meant an amount, concentration or dosage that is capable of treating at least one symptom of the indicated medical condition.

Thus, in one aspect the present invention is drawn to a method for the treatment of a patient having hyperglycemia or hypertriglyceremia and/or elevated levels of blood insulin comprising administering to said patient a therapeutically effective amount of an α2 agonist composition comprising an α2 receptor subtype agonist. In a preferred aspect, the invention comprises administering to a patient a therapeutically effective amount of a non-sedating α2 agonist composition comprising a α2 agonist lacking significant α2A activity.

Compound 1, illustrated below, is an α2 agonist composition that may be used according to the method of the invention: 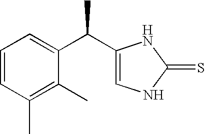
In another aspect the alpha 2-receptor agonist composition comprises a non-sedating α2B selective agonist. By “alpha 2B selective agonist” is meant that i) the efficacy relative to a standard full agonist at the α2B receptor subtype is greater than its efficacy relative to a standard full agonist at the α2A or α2C receptor subtypes and that the relative efficacy at the α2A or α2C receptor subtypes is ≦0.4; or ii) the potency of the compound at the α2B receptor subtype is at least 10 fold greater than at the α2A or α2C receptor subtypes under the same experimental conditions.

In another embodiment of the invention, the non-sedating α2B selective agonist has a chemical structure chosen from:

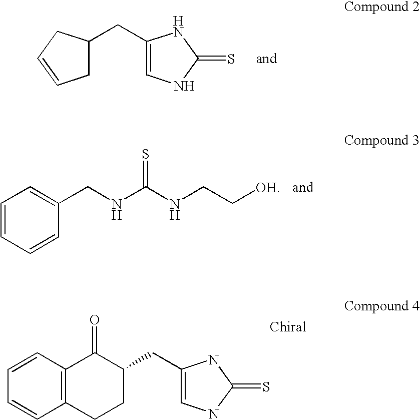

It is important to note that compounds 1, 2, and 4 are of the imidazole-2-thione class of compounds, while compound 3 belongs to the thiourea chemical class; thus the methods and compositions of the present invention are not limited by structure, but apply equally to all alpha 2 non-sedating compounds. Such compounds have now been characterized in the imidazole, thiourea, imidazoline, and imidazole thione chemical classes. Additional chemical classes which comprise non-sedating α2 receptor agonists may include, without limitation, the phenethylamine, amino thiazine, amino imidazoline, benzazepine, amino oxazoline, amino thiazoline, quinazoline, guanidine, piperazine, yohimbine alkaloid, and phenoxypropanolamine chemical classes.

As is well known in the art, sedation is a term that means a reduction in motor activity. The phrase “without concomitant sedation”, or “non-sedating” as used herein in reference to a α2 selective or α2 pan agonist, means that, upon administration, the agonist produces less than about 30% sedation at a dose at least 10-fold greater than the dose of selective agonist required to reduce blood glucose in a hyperglycemic mammal by 20% or more. For example, an α2-selective agonist is administered to a mammal at a dose of 2 mg/kg and reduces blood glucose from 250 mg/dl to 200 mg/dl; the α2-selective agonist is “non-sedating” if it produces less than about 30% sedation when administered to the mammal at a dose of at least about 20 mg/kg. The amount of α2 receptor agonist required to reduce blood glucose by 20% or more will generally be a “therapeutically effective dose,” although in certain circumstances a lower reduction (e.g., 10%) may be desirable.

Thus as used herein the term “non-sedating” or “without concomitant sedation” does not mean that the indicated compound lacks sedative activity at any dosage; rather it is always an indication of lack of sedation relative to a therapeutically effective dose.

As non-limiting examples, the dose of the non-sedating α2 agonist required to produce about 30% sedation (reduction in motor activity) can be at least 25-fold greater than, 50-fold greater than, 100-fold greater than, 250-fold greater than, 500-fold greater than, 1000-fold greater than, 2500-fold greater than, 5000-fold greater than, or 10,000-fold greater than less than the dose of the same α2 agonist required to produce a reduction of blood glucose in a hyperglycemic mammal to 110 mg/dl or less. Methods for determining the extent of a reduction in blood glucose, as well as the extent of sedation are described herein and further are well known in the art.

In additional embodiments, the present invention may comprise a composition having anti-hyperglycemic activity comprising a non-sedating α2-receptor agonist present at a dosage effective to deliver a therapeutically effective dosage of said agent when administered to a mammal in need thereof.

Generally, methods of administering a drug may include any means sufficient to deliver an effective dose of the agent. Thus, preferred routes of administration for the α2 agonist composition of the invention may be peripheral or non-peripheral and include oral, intravenous, intrathecal and epidural administration. Other possible means of administration of the non α2 agonist composition include, without limitation, by intrathecal pump, subcutaneous pump, dermal patch, intravenous injection, subcutaneous injection, intramuscular injection, and an oral pill, or a combination of such methods. While peripheral means of administration of the non-sedating α2 agonist composition are not currently preferred in the treatment of hyperglycemia or hyperlipidemia, the advantages of the instantly claimed methods may be observed in such cases as well, depending at least in part on the bioavailability of the agent or agents comprised in the α2 agonist composition.

It is understood that the pharmaceutical compositions comprising the α2 agonist composition useful in the present invention optionally (but preferably) includes an excipient such as a pharmaceutically acceptable carrier or a diluent, which is any carrier or diluent that has substantially no long term or permanent detrimental effect when administered to a subject. An excipient generally is mixed with the active compound(s), or permitted to dilute or enclose the active compound(s). A carrier can be a solid, semi-solid, or liquid agent that acts as an excipient or vehicle for the active compound. Examples of solid carriers include, without limitation, pharmaceutical grades of mannitol, lactose, starch, magnesium stearate, sodium saccharin, polyalkylene glycols, talcum, cellulose, glucose, sucrose and magnesium carbonate. Suppository formulations can include, for example, propylene glycol as a carrier. Examples of pharmaceutically acceptable carriers and diluents include, without limitation, water, such as distilled or deionized water; saline; aqueous dextrose, glycerol, ethanol and the like. It is understood that the active ingredients can be soluble or can be delivered as a suspension in the desired carrier or diluent, depending upon the means of administration.

The α2 agonist compositions may also optionally include one or more agents such as, without limitation, emulsifying agents, wetting agents, sweetening or flavoring agents, tonicity adjusters, preservatives, buffers or anti-oxidants. Tonicity adjustors useful in a pharmaceutical composition include, but are not limited to, salts such as sodium acetate, sodium chloride, potassium chloride, mannitol or glycerin and other pharmaceutically acceptable tonicity adjustors. Preservatives useful in pharmaceutical compositions include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in pharmaceutical compositions are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition useful in the methods of the invention. See, for example, Remington's Pharmaceutical Sciences Mack Publishing Company, Easton, Pa. 16.sup.th Edition 1980. Furthermore, an α2 agonist composition may be administered in conjunction with one or more other therapeutic substances, in the same or different pharmaceutical composition and by the same or different routes of administration.

The active agents in the α2 agonist composition are administered in an effective amount. Such an effective amount generally is the minimum dose necessary to achieve the desired prevention or reduction in severity of hyperglycemia or hyperlipidemia. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity and type of stress-associated condition, the age and weight of the patient, the patient's general physical condition, and the pharmaceutical formulation and route of administration. Suppositories and extended release formulations also can be useful in the methods of the invention, including, for example, dermal patches, formulations for deposit on or under the skin and formulations for intramuscular injection.

A pharmaceutical composition useful in the methods of the invention can be administered to a subject by a variety of means depending, for example, on the type of condition to be treated, the pharmaceutical formulation, and the history, risk factors and symptoms of the subject. Routes of administration suitable for the methods of the invention include both systemic and local administration. As non-limiting examples, a pharmaceutical composition useful in the method of the invention can be administered orally; parenterally; by pump, for example a subcutaneous pump; by dermal patch; by intravenous, intra-articular, subcutaneous or intramuscular injection; by topical drops, creams, gels or ointments; as an implanted or injected extended release formulation; by subcutaneous minipump or other implanted device; by intrathecal pump or injection; or by epidural injection. Depending on the mode of administration, the α2 agonist composition can be incorporated in any pharmaceutically acceptable dosage form such as, without limitation, a tablet, pill, capsule, suppository, powder, liquid, suspension, emulsion, aerosol or the like, and can optionally be packaged in unit dosage form suitable for single administration of precise dosages, or sustained release dosage forms for continuous controlled administration.

A method of the invention can be practiced by peripheral administration of the α2 agonist composition. As used herein, the term “peripheral administration” or “administered peripherally” means introducing the α2 agonist composition into a subject outside of the central nervous system. Peripheral administration encompasses any route of administration other than direct administration to the spine or brain.

Peripheral administration can be local or systemic. Local administration results in significantly more of a pharmaceutical composition being delivered to and about the site of local administration than to regions distal to the site of administration. Systemic administration results in delivery of a pharmaceutical composition essentially throughout at least the entire peripheral system of the subject.

Routes of peripheral administration useful in the methods of the invention encompass, without limitation, oral administration, topical administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

Each and every published patent, patent application publication and other reference cited in the present application are hereby incorporated by reference as part of this specification.

While this invention has been described with respect to various specific examples and embodiments, it is to be understood that the invention is not limited thereto and that it can be variously practiced within the scope of the following claims.

### BRIEF DESCRIPTION OF THE DRAWINGS

FIG. 1 is a graph showing an increase in body weight of prediabetic female Zucker rats given vehicle or selected non-sedating α2 agonist compositions from initiation (week 7) to end of the study (week 15). At the 8th week, animals were given high fat diet to raise blood glucose.

FIG. 2 is a graph showing the effects on blood glucose levels (at weeks 7, 12 and 15) of chronic treatment of prediabetic Zucker rats with either vehicle or non-sedating α2 agonist compositions.

FIG. 3 is a graph showing the effects on blood triglyceride levels (at weeks 7, 12 and 15) of chronic treatment of prediabetic Zucker rats with either vehicle or non-sedating α2 agonist compositions.

FIG. 4 is a graph showing an increase in body weight of prediabetic female db/db mice given vehicle or selected non-sedating α2 agonist compositions from age=week 6 to the end of the study (week 11).

FIG. 5 is a graph showing the effects on blood glucose levels (at weeks 6, 7, 9 and 11) of chronic treatment of prediabetic db/db mice with either vehicle or non-sedating α2 agonist compositions.

FIG. 6 is a graph showing an increase in body weight of prediabetic female Zucker rats given vehicle or selected non-sedating α2 agonist compositions different from those in Example 1, from initiation (week 8) to end of the study (week 14). At the 9th week, animals were given high fat diet.

FIG. 7 is a graph showing the effects on blood glucose levels (at weeks 8, 12 and 14) of chronic treatment of prediabetic Zucker rats with either vehicle or non-sedating α2 agonist compositions different from those in Example 1.

FIG. 8 is a graph showing the effects on blood triglyceride levels (at weeks 8, 12, and 14) of chronic treatment of prediabetic Zucker ratswith either vehicle or non-sedating α2 agonist compositions different from those in Example 1.

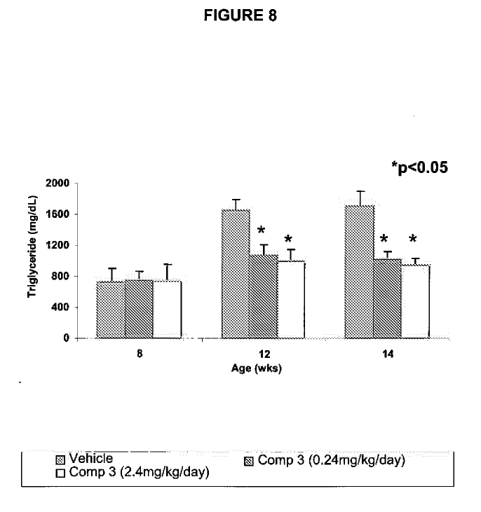

FIG. 9 is a graph showing the effects on blood glucose levels (at weeks 8, 9, 10, 12 and 14) of chronic treatment of diabetic Zucker rats with either vehicle or non-sedating α2 agonist compositions (Compound 1).

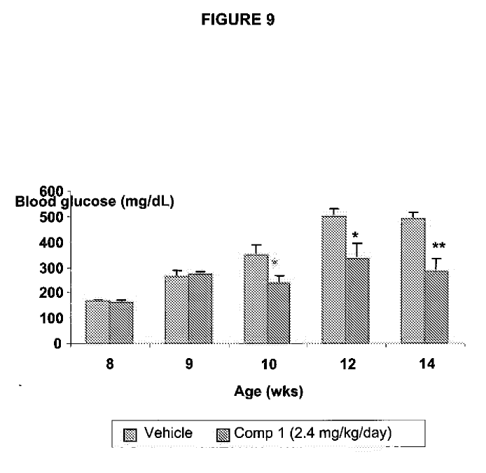

FIG. 10 is a graph showing the effects on blood triglyceride levels (at weeks 8, 10, 12 and 14) of chronic treatment of diabetic Zucker rats with either vehicle or non-sedating α2 agonist compositions (Compound 1).

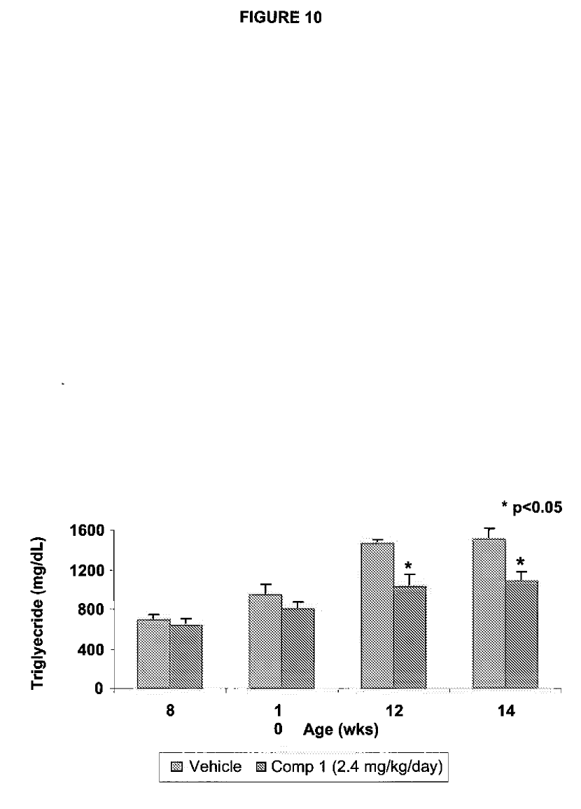

FIG. 11A shows a comparison of insulin levels of female Zucker rats given vehicle control versus those given Compound 1 on week 14.

FIG. 11B shows a comparison of cholesterol levels of female Zucker rats given vehicle control versus those given Compound 1 on week 14.

FIG. 11C shows a comparison of low density lipoprotein (LDL) levels of female Zucker rats given vehicle control versus those given Compound 1 on week 14.

FIG. 11D shows a comparison of glucose levels of female Zucker rats given vehicle control versus those given Compound 1 on week 14.

FIG. 11E shows a comparison of high density lipoprotein (HDL) levels of female Zucker rats given vehicle control versus those given Compound 1 on week 14.

FIG. 11F shows a comparison of FFA (free fatty acid) levels of female Zucker rats given vehicle control versus those given Compound 1 on week 14.

FIG. 12A shows a line graph showing a comparison of blood glucose levels of female Zucker diabetic fatty rats following a single injection of either vehicle or Compound 3.

FIG. 12B shows a line graph showing a comparison of blood glucose levels of female Zucker diabetic fatty rats following a single injection of either vehicle or Compound 1.

FIG. 12C shows a line graph showing a comparison of blood glucose levels of female diabetic Zucker rats following a single injection of either vehicle or Compound 2.

### EXAMPLES

### Example 1

### Chronic Study

Female Zucker rats are animal models for Type II diabetes, developing hyperglycemia and hypertriglyceremia after 1 to 2 weeks of being placed on the high fat diet.

Female Zucker fatty rats (Charles River Laboratories) between 6-7 weeks old were acclimated to the animal research facilities for at least one week. Animals were housed and maintained on a normal diet during the acclimation period.

After acclimation, the rats were weighed and tail-snip glucose and triglyceride levels were determined using a One Touch Ultra® BloodGlucose Monitoring system (LIFESCAN, Milpitas, Calif.) and CardioChek® A analyzer (Polymer Technology Systems, Inc, Indianapolis, Ind.), respectively. The resulting data were used as a baseline for comparison with later treatment results. The animals were randomized to various treatment groups based on blood glucose, triglycerides and body weight.

Vehicle (60% Polyethylene Glycol 300; hereinafter “PEG 300”) or the indicated doses of the tested non-sedating α2 agonists (Compound 1, Compound 2 or Compound 3 in 60% PEG 300) was administered continuously in the experimental rats using an osmotic pump (Alzet Osmotic Pumps, Model 2ML2 (5 μl/hr) Duret Corp., Cupertino, Calif.), which was inserted subcutaneously on back of the animals. Rats were anesthetized by isoflurane inhalation (using 5% isoflurane for induction and 2-3% isoflurane for maintenance of anesthesia by nose cone). An area of approximately six inches² located on the back of each rat was shaved, rinsed with saline solution, cleaned with antiseptic soap solution and wiped with 70% ethanol.

A single 1 inch incision was made perpendicular to the long axis of the animal in the skin covering the lumbar region of the back. Using blunt scissors, a subcutaneous pocket was made toward the head of the animal. A sterile osmotic pump filled with 2 ml of the vehicle or non-sedating α2 agonist composition containing from 0.13 to 6.6 μg/pl of Compound 1, Compound 2, or Compound 3 was placed into the subcutaneous pocket, and the incision was closed using surgical clips.

Compound 1 was administered at 100 μg or 2.4 mg/kg/day. Compound 2 and Compound 3 were administered at 240 μg or 2.4 mg/kg/day. Non-sedating α2 agonists were administered one week prior to the initiation of a high fat diet to the rats, simulating a “pre-diabetic” condition; this diet was continued until the end of the study.

In the second set of experiments (see Example 4), Compound 1 was first administered 1 to 2 weeks after the introduction of the high fat diet to the animals, which continued until end of the study. After 1 or two weeks on the high fat diet, the female Zucker rats become diabetic, with blood glucose at or above 200 mg/dl in the absence of any added therapeutic agent.

For pre-diabetic animals, body weight, blood glucose and triglycerides of the animals were measured as described above at different times after treatment with agonists, high fat diet or both.

Data were compiled and analyzed using Microsoft Excel. Data are expressed as mean +/− standard error of the mean. Comparisons between groups were made using two-tailed, 2-sample equal variance (homoscedastic) student's t-test. The significance values were set at p<0.05 and p<0.01 as indicated by * and **, respectively.

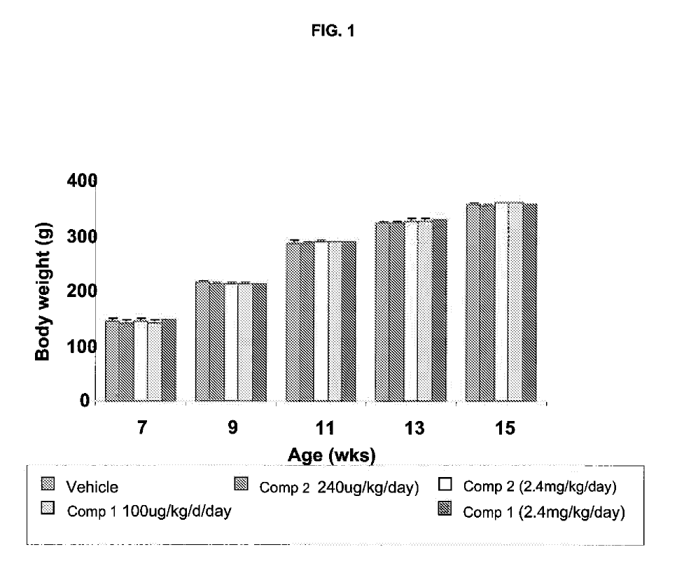

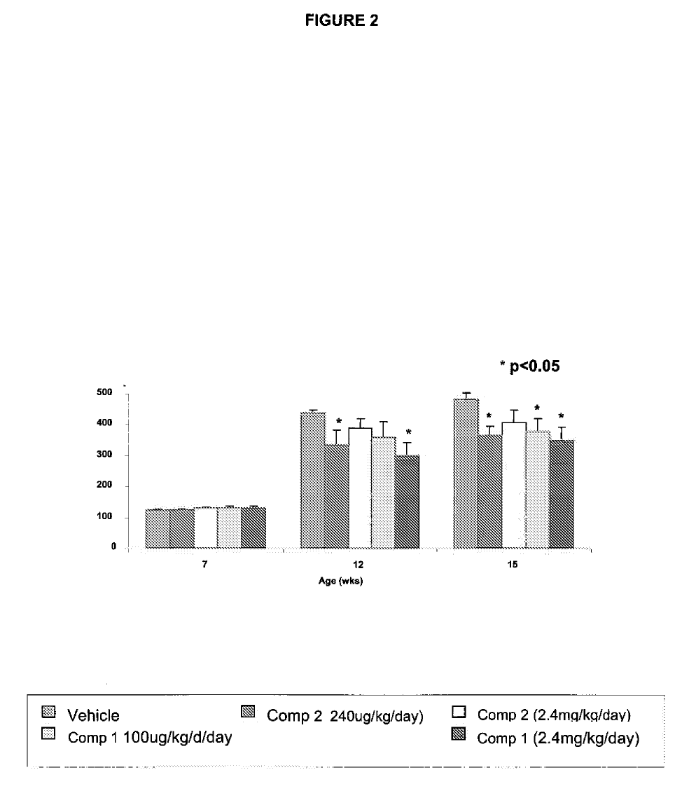

For prediabetic animals, FIG. 1 shows that the body weight of the Zucker rats fed the high fat diet increased with time over the period of the study (from age 7 weeks to age 15 weeks), and that the administration of the non-sedating α2 agonists and vehicle control had no effect on this increase in body weight. Also as expected, the increase in body weight correlated with an approximately four-fold increase in blood glucose levels (from about 100 mg/dl to about 400 mg/dl by week 12 in rats given vehicle alone, with even higher levels seen at week 15 (FIG. 2). FIG. 2 alsodemonstrates that among prediabetic Zucker rats given non-sedating α2 agonist compositions (Compound 2, an α2B selective agonist lacking substantial α2A activity, at 240 μg/kg/day and 2.4 mg/kg/day, and Compound 1, an α2 pan-agonist lacking substantial al activity, at 100 μg/kg/day and 2.4 mg/kg/day, all showed a significant inhibition in the increase in blood glucose relative to the untreated group, with the higher dose of Compound 1 showing the best activity among the groups, lowering blood glucose to about 300 mg/dl at 12 weeks.

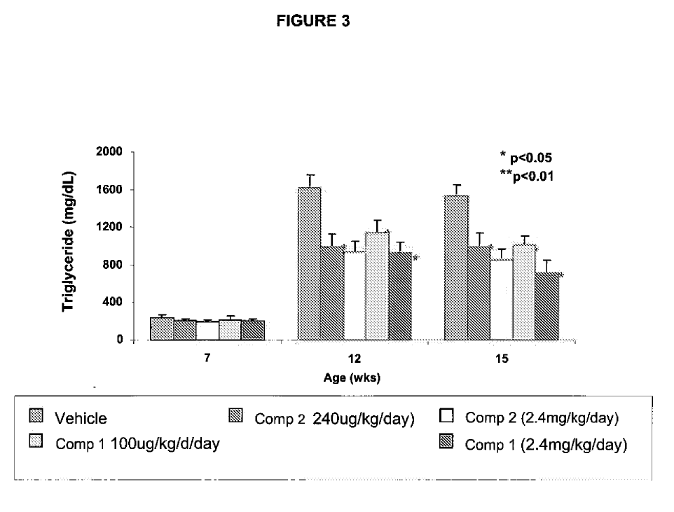

A similar trend was seen in triglyceride levels. FIG. 3 shows that blood triglycerides increased approximately eight-fold in the untreated rats by 12 weeks. All of the dosages of both non-sedating μ2 agonist compositions tested (Compound 1 and Compound 2) significantly prevented in increase in serum triglyceride levels in the prediabetic Zucker rats, with the higher dosage of Compound 1 again showing the best activity among those therapeutic compositions tested.

### Example 2

### Prediabetic db/db Mice

Female db/db mice are considered to be animal models for Type II diabetes and hypertriglyeremia. These animals are identifiably obese at around 3 to 4 weeks of age. Elevation in plasma insulin occurs at about 10 to 14 days of age and elevation of blood sugar at about 4-8 weeks. These animals carry the db gene, which contains a G to T point mutation for the leptin receptor.

Five-week-old female db/db mice (Jackson Laboratories) were acclimated to the animal research facilities for one week and housed and maintained on a normal diet until the initiation of the experiment.

For the study of prediabetic db/db mice after acclimation for one week, the mice (6 weeks old) were weighed and tail-snip glucose levels were determined using One Touch Ultra Blood Glucose Monitoring system (LIFESCAN, Milpitas, Calif.). The animals were randomized into vehicle, clonidine and Compound 2 groups based on blood glucose and body weight. Body weight and blood glucose of db/db mice at week 6 were considered as base-line values.

Once the blood glucose levels of the experimental mice mice were above 150 mg/dL in week 6, vehicle (60% Polyethylene Glycol 300, PEG 300), clonidine or Compound 2 in 60% PEG 300 was administered continuously using osmotic pumps (Alzet mini-osmotic pumps Model 2002 (0.5 μl/hr), Duret Corp., Cupertino, Calif.), which were inserted subcutaneously on back of the animals. The mice were anesthetized by isoflurane inhalation (5% induction and 2-3% maintenance by nose cone). An area of approximately 1 inch by 1 inch located in the back of the mice was shaved, rinsed with saline solution, cleaned with antiseptic soap solution and wiped with 70% ethanol. A single 0.5 inch incision was made perpendicular to the long axis of the animal in the skin covering the lumbar region of the back. Using blunt scissors, a subcutaneous pocket was made toward the head of the animal. The sterile osmotic pump filled with 0.5 ml vehicle, clonidine (0.21-0.29 μg/ul) or Compound 2 (5-7 μg/ul) was placed into the subcutaneous pocket, and the incision was closed with surgical clips. Clonidine and Compound 2 were administered at 100 μg/kg/day and 2.4 mg/kg/day, respectively. At different times after administration of vehicle, clonidine or Compound 2, body weight and blood glucose of the animals were measured as described above. Every two weeks the pumps were replaced with fresh ones and dosing was continued through week 11.

Data were compiled and analyzed using Microsoft Excel. Data are expressed as mean +/− standard error of the mean. Comparisons between groups were made using two-tailed, 2-sample equal variance (homoscedastic) student's t-test. The significance value was set at p<0.05 as indicated by *.

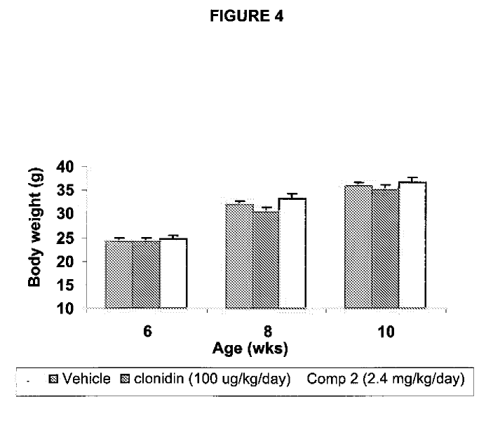

FIG. 4 shows that the body weight of the db/db mice increased steadily from week 6 to week 8 in both control and experimental groups, and similar to the results seen for prediabetic Zucker rats, the increase in body weight of prediabetic db/db mice was unaffected by the administration of 100 μg/kg/day clonidine or 2.4 mg/kg/day of Compound 2. Clonidine, a sedating α2 agonist and 11 imidizole receptor agonist, is known to have hypoglycemic activity and was used as a reference.

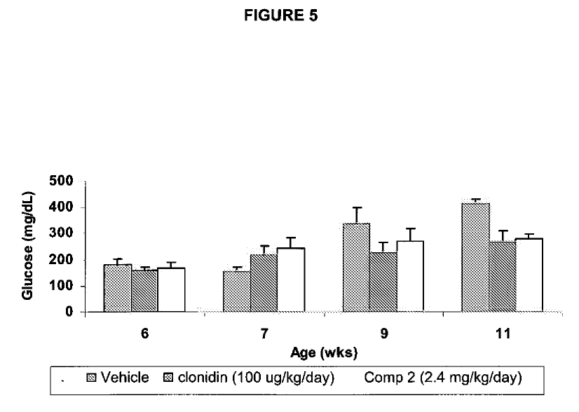

FIG. 5 shows that both clonidine and Compound 2 decreased blood glucose in db/db mice beginning at week 9, and that this trend remained until week 11, the end of the study.

These results and the Zucker rat results (Example 1) indicate that pre-treatment with α2 agonists attenuates the spontaneous increase in blood glucose in pre-diabetic db/db mice, and also in Zucker rats subsequently given a high fat diet. Thus the non-sedating α2 agonist compositions are effective in preventing or lessening the extent of hyperglycemia and hypertriglycermia in prediabetic animals.

### Example 3

### Prediabetic Zucker Rats and Compound 3

Female Zucker rats were handled essentially as described in Example 1.

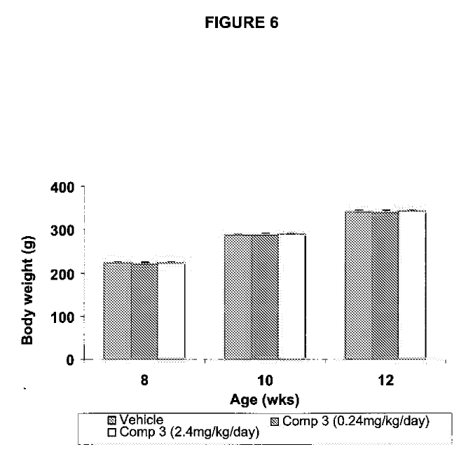

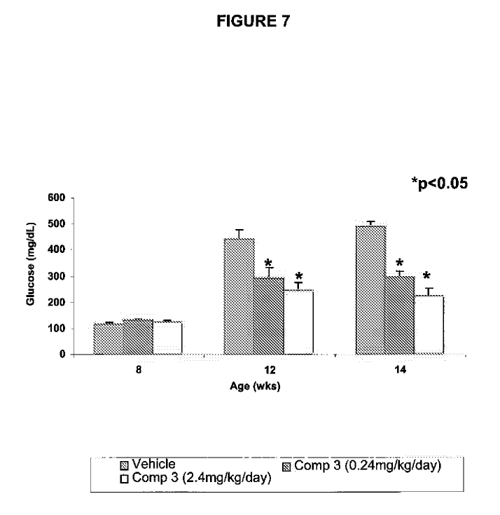

Both Compound 1 and Compound 2 belong to the imidazole-2-thione class of compounds. To determine whether the prophylactic antihyperglycemic and antihypertriglyceremic effect of the non-sedating α2 agonist compositions is limited to certain classes of compounds, Compound 3, a benzyl thiourea having α2B selective activity was tested in the same manner as Compound 1 and Compound 2 at 0.24 mg/kg/day or 2.4 mg/kg/day. FIG. 6 shows no effect on the increase in body weight; FIGS. 7 & 8 show a dose-dependent inhibition in the development of hyperglycemia and hypertriglyceremia, similar to that seen in the cases of Compounds 1 and 2. Thus the prophylactic effect of the non-sedating α2 agonists is unlimited by a particular class of chemical compound.

### Example 4

### Diabetic Zucker Rats; Compound 1

Female Zucker rats were handled essentially as described in Example 1 with the following modifications. The non-sedating α2 agonist composition (Compound 1 at 2.4 mg/kg/day) was administered by osmotic pump at week 9, 1 week after initiation of the diabetic phenotype (week 8) by switching the Zucker rats to a high fat diet. At week 9 the Zucker rats showed a blood glucose level of over 250 mg/dl, indicating that the rats are diabetic. Administration of the drug was continued until week 14. Tail-snip glucose and triglycerides were measured at weeks 8, 9, 10, 12 and 14.

At the end of the experiment (after 14 weeks) the rats were fasted overnight, anesthetized with isofluorane and approximately 2 ml of blood was drawn from the orbital sinus using capillary tubes to measure blood glucose, insulin, triglycerides, cholesterol, HDL, LDL and free fatty acids using an automated clinical chemistry analyzer. Blood glucose, insulin, free fatty acids (FFA), cholesterol, HDL and LDL levels were measured at this point.

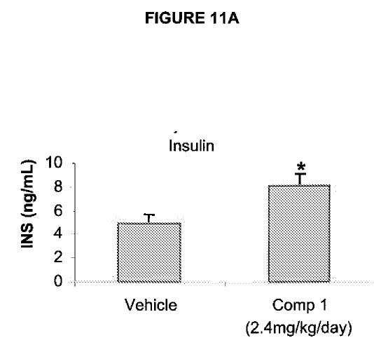

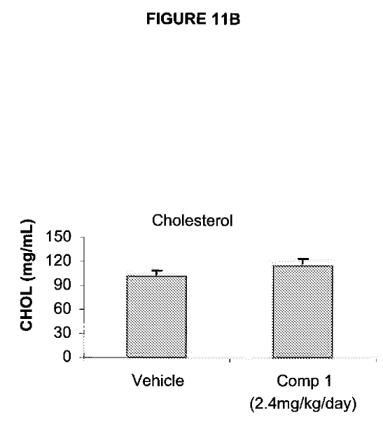

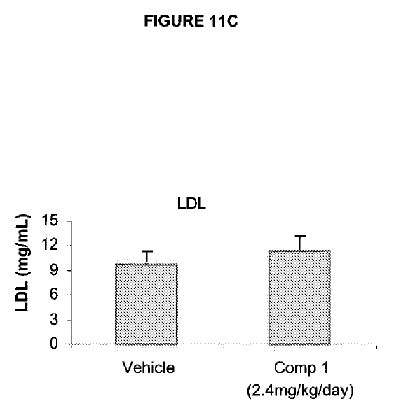

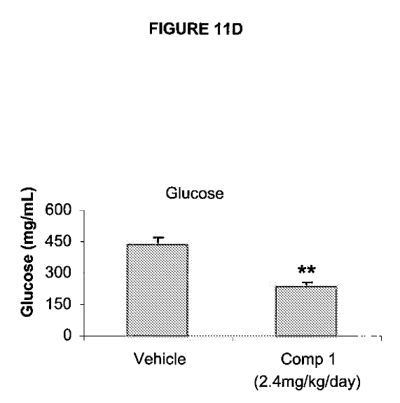

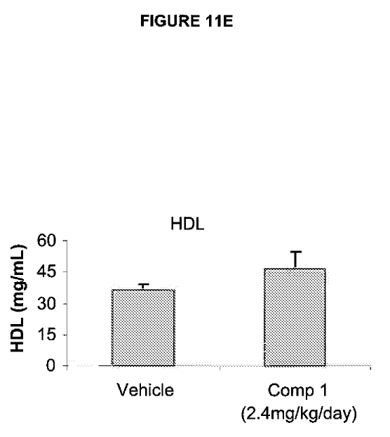

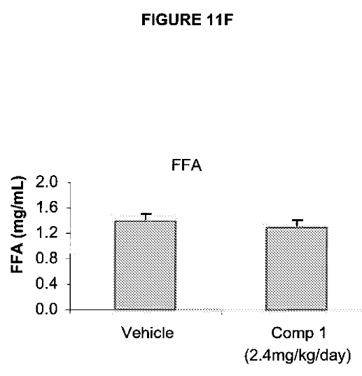

FIG. 11 shows that Compound 1 significantly inhibited the increase in blood glucose of treated diabetic animals relative to those treated with vehicle alone. The Compound 1 treated group also had a significantly increased level of serum insulin. However, no effect on blood cholesterol, FFA, HDL and LDL was seen. Lean Zucker rats were unaffected by the non-sedating α2 agonist compositions.

### Example 5

### Female Zucker Rats: Acute Treatment

The following experiment was performed to see whether chronic treatment (such as by osmotic pump) is necessary to observe the anti-diabetic effects of the non-sedating α2 agonist compositions.

Female Zucker fatty rats were fed the high fat diet for 3 to 4 weeks to raise their blood glucose levels above 300 mg/dl. The animals were then fasted overnight. The following morning blood glucose was measured; this baseline point was called 0 hr. Animals were then treated with vehicle (60% PEG 300 in water) or a single injection of a non-sedating α2 agonist composition (Compound 1, Compound 2 andCompound 3, respectively) in the vehicle (300 μg/kg) using intraperitoneal (IP) injection. Blood glucose was measured at 1, 3 and 5 hrs after IP injection.

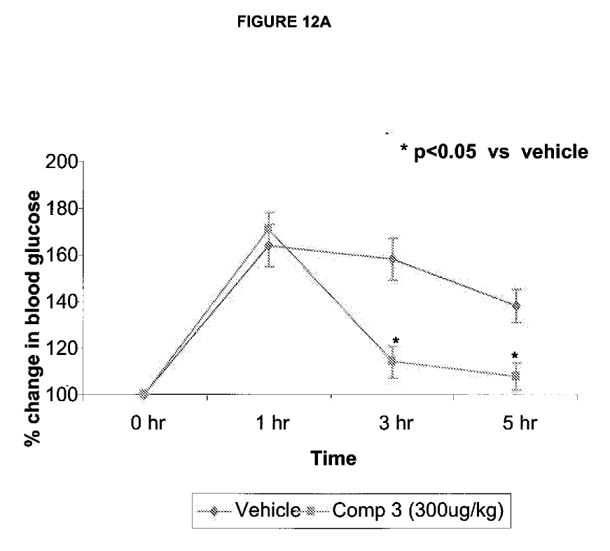

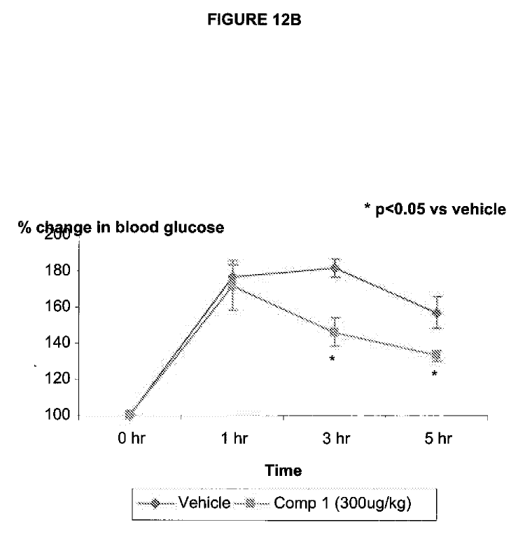

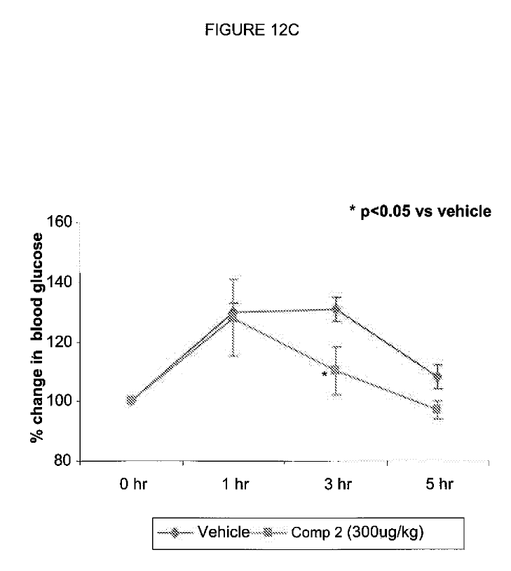

FIG. 12A shows a time course of changes in blood glucose levels in Zucker rats following injection of 300 μg/kg Compound 3. FIG. 12B shows a time course of changes in blood glucose levels following injection of 300 μg/kg Compound 1. FIG. 12C shows a time course of changes in blood glucose levels following injection of 300 μg/kg Compound 2.

In all cases injection of the non-sedating α2 agonist composition resulted in a significant decrease in blood glucose levels relative to the vehicle only control animals.

While this invention has been described with respect to various specific examples and embodiments, it is to be understood that the invention is not limited thereto and that it can be variously practiced within the scope of the following claims.

What is claimed is:

1. A method of treating at least one symptom of diabetes in a mammal, the method comprising the step of administering to the mammal a therapeutically effective amount of an α2 agonist composition

2. The method of claim 1 wherein the symptom comprises hyperglycemia.

3. The method of claim 1 wherein the symptom comprises hypertriglyceridemia.

4. The method of claim 1 wherein the symptom comprises increased levels of blood insulin.

5. The method of claim 1 wherein the symptom comprises hyperlipidemia,

6. The method of claim 1 wherein the α2 agonist composition comprises an α2 agonist lacking significant α1 activity.

7. The method of claim 6 wherein the α2 agonist is an α2 pan-agonist.

8. The method of claim 7 wherein the α2 pan-agonist is the sole active agent in the non-sedating α2 agonist composition.

9. The method of claim 6 wherein the α2 agonist lacks significant α2A activity.

10. The method of claim 1 wherein the α2 agonist compositioncomprises an α2 agonist lacking significant α2A activity.

11. The method of claim 10 wherein the α2 agonist is an α2B selective agonist.

12. The method of claim 1 wherein the α2 agonist composition is non-sedating.

13. The method of claim 11 wherein the α2 agonist comprises Compound 2.

14. The method of claim 11 wherein the α2 agonist comprises Compound 3.

15. The method of claim 1 wherein the α2 agonist composition comprises an α2 pan-agonist.

16. The method of claim 11 wherein the α2 pan-agonist comprises Compound 4.

17. The method of claim 1 wherein the α2 agonist composition comprises an α1 antagonist.

18. The method of claim 1 wherein the α2 agonist composition comprises an alpha 2A antagonist.

19. The method of claim 1 wherein the α2 agonist composition is administered by injection.

20. The method of claim 1 wherein the α2 agonist composition is administered orally.

21. The method of claim 1 wherein the α2 agonist composition isadministered by means of a pump.

22. The method of claim 1 wherein the α2 agonist composition is administered by means of a transdermal patch.
